(19) United States
(12) Patent Application Publication
(10) Pub. No.: US 2016/0213280 A1
(43) Pub. Date: Jul. 28, 2016

(54) MEDICAL DEVICE FOR CONTACT SENSING

(71) Applicant: Boston Scientific Scimed Inc., Maple Grove, MN (US)

(72) Inventors: Jeffrey A. Sarge, Fremont, CA (US); Darrell L. Rankin, Milpitas, CA (US); John C. Potosky, San Jose, CA (US)

(21) Appl. No.: 15/004,859

(22) Filed: Jan. 22, 2016

Related U.S. Application Data
(60) Provisional application No. 62/107,176, filed on Jan. 23, 2015.

(51) Int. Cl. A61B 5/06; A61B 5/00

(52) U.S. Cl. CPC A61B 5/068; A61B 5/6852; A61B 5/742; A61M 2025/0166

(57) ABSTRACT

An example system for sensing catheter contact is disclosed. The system includes an elongate tubular member, a tip member and a flexible support structure including a support member. A proximal portion of the support structure may be coupled to the distal end of the tubular member while a distal portion may be coupled to the proximal end of the tip member. The system may also include an electroactive polymer member disposed along the support member, wherein displacement of the support member activates the electroactive polymer member such that an electrical response is output from the electroactive polymer member. A processor, electrically coupled to the electroactive polymer member, may be configured to evaluate the electrical response from the electroactive polymer member to determine a contact force of the tip member with tissue.

### CROSS-REFERENCE TO RELATED APPLICATION

This application claims priority to Provisional Application No. 62/107,176, filed Jan. 23, 2015, which is herein incorporated by reference in its entirety.

### TECHNICAL FIELD

The present disclosure pertains to medical devices, and methods for manufacturing medical devices. More particularly, the present disclosure pertains to elongated intracorporeal medical devices including a tubular member connected with other structures, and methods for manufacturing and using such devices.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example system for sensing catheter contact may include an elongate tubular member having a proximal end and a distal end, a tip member having a proximal end and a distal end and a flexible support structure including a support member, wherein the support structure has a proximal portion and a distal portion, wherein the proximal portion is coupled to the distal end of the elongate tubular member, and wherein the distal portion is coupled to the proximal end of the tip member. The system may also include a polymer member disposed along the support member, the polymer member formed of an electroactive polymer, wherein displacement of the support member activates the polymer member such that an electrical response is output from the polymer member. The system may also include a processor electrically coupled to the polymer member, wherein the processor is configured to evaluate the electrical response output from the polymer member to determine a contact force of the tip member with tissue.

Alternatively or additionally, the flexible support structure may include a first support member, a second support member and a third support member, and wherein the polymer member is disposed along at least one of the first, second and third support members.

Alternatively or additionally, the distal end of the tubular member may have a substantially circular circumference, and wherein the first, second and third support members are spaced from one another around the circumference.

Alternatively or additionally, at least one of the first, second and third support members may include an arcuate bend.

Alternatively or additionally, the first, second and third support members may be configured to flex in response to the contact force.

Alternatively or additionally, the elongate tubular member may include a longitudinal axis, and wherein the first, second and third support members include a proximal portion, an intermediate portion and a distal portion, and wherein the intermediate portions of the first, second and third support members extend inward toward the longitudinal axis relative to the proximal and distal ends of the first, second and third support members.

Alternatively or additionally, the flexible support structure further may comprise a printed circuit board, and wherein the printed circuit board includes the first, second and third support members.

Alternatively or additionally, the printed circuit board may include a hub, and wherein a distal end of the first, second and third support members are coupled to the hub and wherein the proximal end of the first, second and third support members extends away from the hub.

Alternatively or additionally, a polymer member may be disposed along at least one of the first support member, the second support member and the third support member.

Alternatively or additionally, the proximal ends of the first, second and third support members may be coupled to the distal end of the elongate tubular member.

Alternatively or additionally, wherein at least one of the first, second and third support members extends along the elongate tubular member.

Alternatively or additionally, at least one of the first, second and third support members may extend to the proximal end of the elongate tubular member.

Alternatively or additionally, the flexible support structure may further comprise a printed circuit board, and wherein the printed circuit board includes a support member.

Alternatively or additionally, the printed circuit board may have a first layer, and wherein the first layer is an electroactive polymer.

Alternatively or additionally, the flexible support structure may include a helical member, and wherein the polymer member is disposed along the helical member.

Another example system for sensing catheter contact may include a catheter having a proximal end region and a distal end region and a lumen extending therethrough, and wherein the catheter includes a flexible support structure disposed along the distal end region, and wherein the support structure includes a support member, and wherein the support member is configured to deflect in response to a contact force. The system may also include a polymer member disposed along the support member, the polymer member including an electroactive polymer, wherein deflection of the support member activates the polymer member such that an electrical response is output from the polymer member. The system may also include a processor electrically coupled to the polymer member, wherein the processor is configured to evaluate the electrical response output from the polymer member to determine a contact force of the tip member with tissue.

Alternatively or additionally, the flexible support structure may be disposed within the lumen of the catheter.

Alternatively or additionally, the flexible support structure may include a first support member, a second support member and a third support member and wherein a first polymer member, a second polymer member and a third polymer member are disposed along the first, second and third support members.

Alternatively or additionally, at least one of the first polymer member, the second polymer member and the third polymer member is configured to determine the magnitude and/or the direction of the contact force.

Another example system for sensing catheter contact may include an elongate tubular member having a proximal end and a distal end, a tip member having a proximal end and a distal end and a first support member, a second support member and a third support member, wherein the support members have a proximal portion and a distal portion, wherein the proximal portions are coupled to the distal end of the elongate tubular member, and wherein the distal portions are coupled to the proximal end of the tip member. The system may also include a first polymer member disposed along the support member, a second polymer member disposed along the second support member and a third polymer member disposed along the third support member, wherein the polymer members are formed of an electroactive polymer, wherein displacement of the support member activates the polymer members such that an electrical response is output from the polymer members. The system may also include a processor electrically coupled to the polymer members, wherein the processor is configured to sense the electrical response output from the polymer members, evaluate the electrical response to determine a magnitude and direction of the contact force and display the contact force magnitude and/or direction on a display.

The above summary of some embodiments is not intended to describe each disclosed embodiment or every implementation of the present invention. The Figures, and Detailed Description, which follow, more particularly exemplify these embodiments.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

The invention may be more completely understood in consideration of the following detailed description of various embodiments of the invention in connection with the accompanying drawings, in which:

FIG. 1 is a schematic view of an embodiment of a catheter system for accessing a targeted tissue region in the body for diagnostic and therapeutic purposes.

FIG. 2 is a schematic view of an embodiment of a distal region of a sensing catheter having sensing members for use in association with the system of FIG.

FIG. 3 is a cross-section of the sensing members in the system of FIG. 2.

FIG. 4 is a schematic view of an embodiment of a distal region of a sensing catheter including a helical sensing member.

FIG. 5 is a schematic view of an embodiment of a sensing catheter including elongated sensing members.

FIG. 6 is a schematic view of the support structure of the sensing catheter of the system of FIG. 5.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

Treating heart rhythm disorders often involves the introduction of one or more sensing devices into a cardiac chamber to collect and/or map physiological or diagnostic information necessary for subsequent therapies. For example, in order to perform ablation therapy in a cardiac chamber, it may be desirable to use a contact sensing catheter to determine the proximity of the catheter tip to the chamber wall and/or the degree of force applied to adjacent cardiac tissue. However, the forces applied to the sensing catheter, catheter tip and/or catheter sensors may vary depending on the degree to which the catheter system is manipulated within the cardiac chamber. For example, if a clinician applies a significant amount of force to the catheter or other mapping/sensing device, the catheter tip and or sensors may be deflected and/or stressed to a significant degree. Further, the catheter system may be subjected to a wide range of temperatures. In some instances, the force applied to the sensors and or the temperature range may be significant enough to damage the sensors. Therefore, it may be desirable to utilize materials and/or design the sensors such that they can withstand significant forces and/or temperatures, yet maintain the sensitivity necessary to accurately sense desired physiological data. The methods and systems disclosed herein are designed to overcome at least some of the limitations of current sensing catheter design. For example, some of the methods disclosed herein may include utilizing an electroactive polymer (EAP) and/or a derivative thereof (e.g. electromechanical polymers (EMP)) in combination with a catheter support structure to sense contact force and/or proximity to cardiac tissue. Other methods and medical devices are also disclosed.

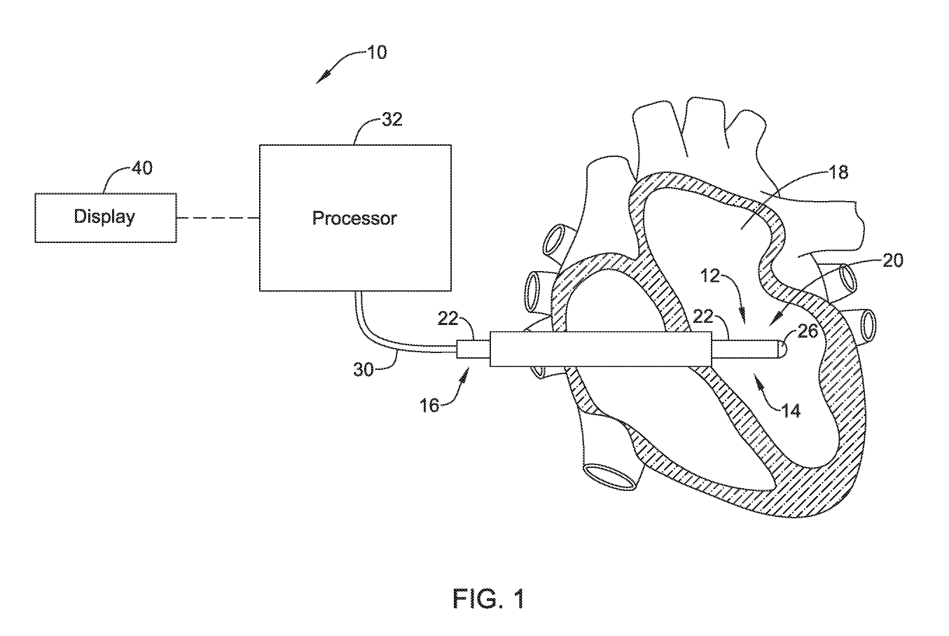

FIG. 1 is a schematic view of a system 10 for accessing a targeted tissue region in the body of a patient for diagnostic and/or therapeutic purposes. FIG. 1 generally shows the system 10 deployed in a region of the heart. For example, system 10 may be deployed in any chamber of the heart, such as the left atrium, left ventricle, right atrium, or right ventricle, another region of the cardiovascular system, or other anatomical region. While the illustrated embodiment shows the system 10 being used for sensing contact and/or proximity to myocardial tissue, the system 10 (and the methods described herein) may alternatively be configured for use in other tissue applications, such as procedures for sensing tissue in the prostrate, brain, gall bladder, uterus, nerves, blood vessels and other regions of the body, including body regions not typically accessed by a catheter.

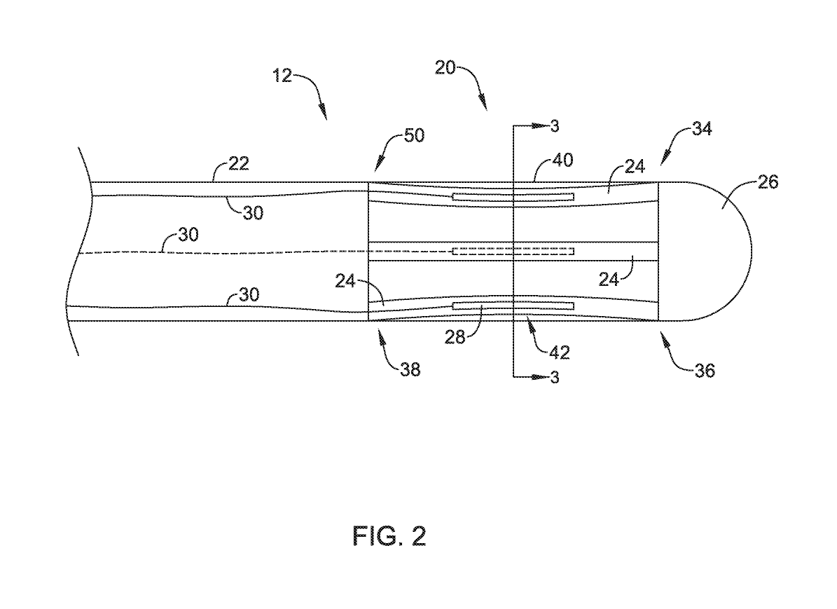

System 10 may include catheter 12. In FIG. 1, catheter 12 may be introduced into the selected heart region 18 through a vein or artery (e.g., the femoral vein or artery) through suitable percutaneous access. The catheter 12 may have a body portion 22, a distal end region 14 and a proximal end region 16. The distal end region 14 of the catheter 12 may include support structure 20 and distal tip 26. Support structure 20 may include a plurality of sensing support elements (not shown in FIG. 1, but shown in FIG. 2). The support elements may be configured to sense physiological activity in the anatomical region. For example, the support elements may be configured to sense contact and/or proximity to anatomical tissue.

In some instances, catheter 12 may be an ablation catheter. In those instances, distal tip 26 may include an ablation electrode. Further, the ablation electrode may be coupled to a system for delivering energy to the ablation electrode. For example, the ablation electrode may be coupled to a RF generator.

In other instances, catheter 12 may be a mapping catheter. In those instances, distal tip 26 may include one or more mapping electrodes. The mapping electrodes may be couple to processor 32. Processor 32 may receive electrical information sensed by the mapping electrodes. Further, processor 32 may process sensed electrical information and output corresponding diagnostic information to display 40.

While the above examples describe catheter 12 being either an ablation catheter or a mapping catheter, it is contemplated that catheter 12 could be some combination of both an ablation catheter and a mapping catheter. Further, distal tip 26 may include both an ablation electrode and one or more mapping electrodes.

Additionally, support structure 20 may be electrically coupled to a processing system 32. Conductive members 30 may be electrically coupled to each sensing support element on support structure 20. The conductive members 30 may extend through catheter body 22 and may electrically couple the sensing elements of support structure 20 to an input of processing system 32, as will be described later in greater detail. The sensing elements may respond to a deflection, an applied force, a change in resistance, or the like. In some instances, the sensing elements may generate an electrical response to a force applied to distal end region 14 of catheter 12 as the distal end region 14 of catheter 12 is advanced into cardiac tissue. The sensed force may be processed by processing system 32 to assist the physician by generating a parameter, e.g., a number, color, texture, audible tone, etc. which corresponds to a diagnostic and/or treatment procedure, e.g. an ablation procedure. The parameter may be used to guide the treatment of a tissue pathology, e.g., ablation therapy.

Processing system 32 includes dedicated circuitry (e.g., discrete logic elements and one or more microcontrollers; application-specific integrated circuits (ASICs); or specially configured programmable devices, such as, for example, programmable logic devices (PLDs) or field programmable gate arrays (FPGAs)) for receiving and/or processing the acquired electrical signals. In some embodiments, processing system 32 includes a general purpose microprocessor and/or a specialized microprocessor (e.g., a digital signal processor, or DSP, which may be optimized for processing activation signals) that executes instructions to receive, analyze and display information associated with the received electrical signals. In such implementations, processing system 32 can include program instructions, which when executed, perform part of the signal processing. Program instructions can include, for example, firmware, microcode or application code that is executed by microprocessors or microcontrollers. The above-mentioned implementations are merely exemplary. Other program instructions are contemplated.

The processing system 32 may output to device 40 the display of relevant parameters (e.g. contact force magnitude or direction) for viewing by a physician. In the illustrated embodiment, device 40 is a display, such as a CRT, LED, or other type of display, or a printer, for example. Device 40 may present the relevant parameters in a format useful to the physician. In addition, the processing system 32 may generate position-identifying output for display on the device 40 that aids the physician in guiding an ablation electrode into contact with tissue at the site identified for ablation. For example, the display may show one or more elements of a force vector corresponding to one or more sensing elements and/or a degree of contact of the distal end region 14 of catheter 12 with tissue. It is contemplated that the elements may be displayed alone or in combination with one another.

FIG. 2 illustrates an embodiment of the distal end region of the catheter 12 which may be suitable for use in system 10 shown in FIG. 1. Catheter 12 may include catheter body 22 and a support structure 20. In some examples, body portion 22 may resemble an elongate tubular member. Further, the terms “body portion” and “elongate tubular member” may be used interchangeably herein. Catheter 12 may also include a distal tip 26 extending distally from distal end 36 of support structure 20. Catheter body 22 may include a lumen extending therethrough (not shown). In some instances, a covering 40 may extend over or along support structure 20. Covering 40 may be a coating, a solid tubular member, a distal end region of catheter body 22 or the like. In some instances, covering 40 may be attached to support structure 20. In other instances, covering 40 may extend from the distal end 50 of catheter body 22.

As depicted in FIG. 2, support structure 20 may include one or more support members 24. For example, while FIG. 2 shows three support members 24, it is understood that the number of support members 24 may be 1, 2, 3, 4, 5, 6, 7, 8, 9, 10, 20 or the like. Support members 24 may have a proximal end 38 and a distal end 36. The proximal end 38 of support member 24 may be coupled to the distal end 50 of catheter body 22, or otherwise fixed relative to the catheter body 22. Similarly, the distal end 36 of support member 24 may be coupled to a proximal end 34 of distal tip 26, or otherwise fixed relative to the distal tip 26. In some instances (for example, as shown in FIG. 2), support member 24 may resemble splines and/or elongated strips extending from the catheter body 22 to the distal tip 26. However, while FIG. 2 depicts support member 24 as an elongated strip, it is contemplated that support member 24 could be a variety of shapes, sizes, geometries or materials. For example, support members 24 could be a single tubular member spanning the gap from the catheter body to the distal tip. Alternatively, support member 24 may be one or more cylindrical rod-shaped members, or other desired configuration.

The embodiment of support structure 20 in FIG. 2 shows three support members 24 arranged around the distal end region 50 of catheter body 22. In some instances, support members 24 may be pre-formed into a desired shape. For example, FIG. 2 shows the support members 24 as including an arcuate bend along the length of the support member. In other words, support members 24 may include intermediate region 42 which extends and/or curves radially inward toward a central longitudinal axis of catheter body 22 relative to proximal and distal ends of the support members 24. The pre-formed shape (e.g. bow, arcuate bend, etc.) may allow the support member 24 to flex and/or deflect in response to an applied force. In other words, the pre-formed shape may provide a natural flexion point in support members 24, thereby reducing the longitudinal stiffness that support members 24 may otherwise have to overcome to flex in response to an applied contact force. While support members 24 have been described above as having an arcuate bend, it is understood that in some instances other shapes (e.g. solid, rigid or straight shapes) may be desirable. For example, in some instances the support members 24 may include a proximal segment converging with a distal segment at a vertex (e.g., point), wherein the vertex or point is located radially inward toward the central longitudinal axis of catheter body 22 relative to proximal and distal ends of the support members 24.

Additionally, FIG. 2 shows polymer members 28 disposed along support members 24. In some instances, polymer members 28 may be affixed along support members 24. Further, it is contemplated that polymer members 28 may be coupled to support members 24 using a variety of methodologies. For example, polymer members 28 may be fixedly secured or bonded along a surface of the support members 24. As discussed herein, polymer members 28 may be described as being positioned “along,” “on,” and/or otherwise embedded and/or encased on any structure contemplated herein. This is not intended to be limiting. Rather, polymer members 28 can be positioned and/or otherwise located at any suitable position/location along support structure 20, support members 24, along catheter 12 and/or any other catheter structure. Positioning/locating polymer members 28 may include embedding, partially embedding, encasing, partially encasing, isolating, attaching, affixing, fastening, bonding to the outer surface, embedding within the wall, or the like. Polymer member 28 may include a variety of different materials. For example, polymer member 28 may include polyvinylidene fluoride.

Polymer member 28 may be an electroactive polymer (EAP) including any derivative or subgroup thereof (e.g. electromechanical polymers (EMP)). The term EAP is not meant to be limiting. Rather, as used with any disclosed embodiment herein, the term EAP encompasses not only electroactive polymers, but also any derivative or subgroup thereof (e.g. electromechanical polymers (EMP)). EAP may provide significant benefits when incorporated into the design of contact sensing catheters. For example, EAP may be able to withstand a significant degree of deflection and temperature gradients and still output an electrical response that is capable of being received by processing system 32 and processed into beneficial diagnostic information.

In some instances, an EAP may output an electrical signal and/or surface charge in response to a change in the polymer's shape, resistance, mechanical properties, or the like. For example, in some instances the contact catheter's distal end region may bend or deflect as a clinician manipulates the catheter inside a heart chamber. The deflection may result in a deflection of one or more of the support members 24. In instances where an EAP is affixed directly to support members 24, deflection of support members 24 may cause a corresponding deflection in the EAP. The deflection of the EAP may cause the EAP to output an electrical signal to processor 32. Processor 32 may receive the electrical signal transmitted from the EAP.

In some instances, an electrical signal output by polymer member 28 may travel along conductive member 30 to processor 32. A separate conductive member 30 may be electrically coupled to each polymer member 28 to transmit separate electrical output signals from each polymer member 28 to processor 32. Conductive member 30 may include a variety of conducting materials (e.g. copper, gold, etc.) or the like. As shown in FIG. 2, conductive member 30 may be coupled to proximal end region 38 of support member 24. In some instances, conductive member 30 may be coupled to the support member 24, polymer member 28, or both. Conductive member 30 may travel along catheter body 22 and terminate at processor 32. It is contemplated that conductive member 30 may be embedded within the wall of catheter body 22, extend through a lumen of catheter body 22, extend along the outside of catheter body 22 or some combination thereof. Other configurations are contemplated.

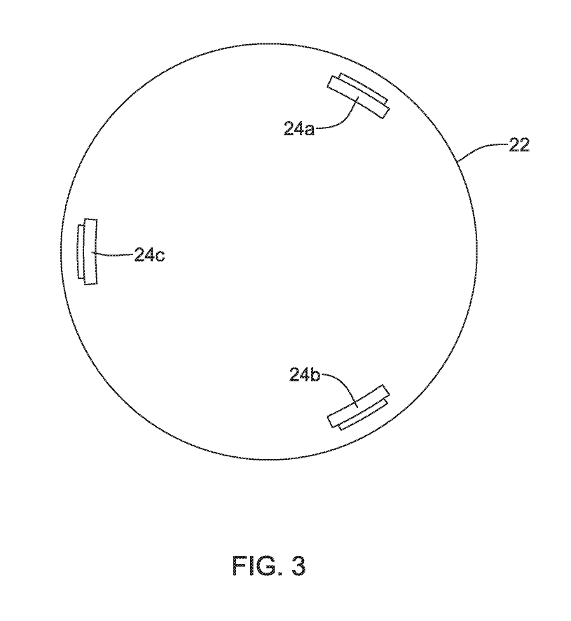

As stated above, support structure 20 may be disposed around the distal end 50 of catheter body 22 in a manner that may be advantageous to determine both the direction and the magnitude of an applied contact force. For example, FIG. 3 shows a cross sectional view along line 3-3 of FIG. 2. FIG. 3 depicts support members 24a, 24b and 24c arranged around the circumference of catheter body 22. For example, a plurality of support members 24, such as three support members 24, may be uniformly or symmetrically arranged around the circumference of catheter body 22. While FIG. 3 shows support members 24 disposed around the outer circumference of catheter body 22, it is contemplated that support members 24 may be disposed at any position around of the outer or inner surface of catheter body 22.

As shown, support members 24a, 24b and 24c are generally arranged in a manner that allows a desired degree of spacing between individual members 24. While FIG. 3 shows support members 24 spaced substantially equidistant from one another (e.g. about 120 degrees), it is contemplated that support members 24 may be spaced in any variety of configurations. For example, the spacing between the members may be any known equidistant or non-equidistant spacing. In some instances, it may be beneficial to utilize three support members, as three support members may permit efficient triangulation of the direction of an applied contact force. However, it is understood that a variety of algorithms, processing, etc. may be employed with fewer or more support members 24 in order to calculate the direction of an applied force.

As discussed above, in some embodiments, support members 24 may be constructed from variety of materials (alone or in combination). For example, support members 24 may be made of a resilient inert material, such as metal (e.g. Nitinol, stainless steel, etc.), silicone rubber, polymer, etc. These materials may provide beneficial characteristics that allow support members 24 to be connected between the catheter body 22 and the distal tip 26 in a resilient, yet flexed condition (e.g. permitting flexible members 24 to bend and conform to an applied contact force). In some instances, the support members 24 may be formed of an electrically conductive material, while in other instances the support members 24 may be formed of an electrically insulative material.

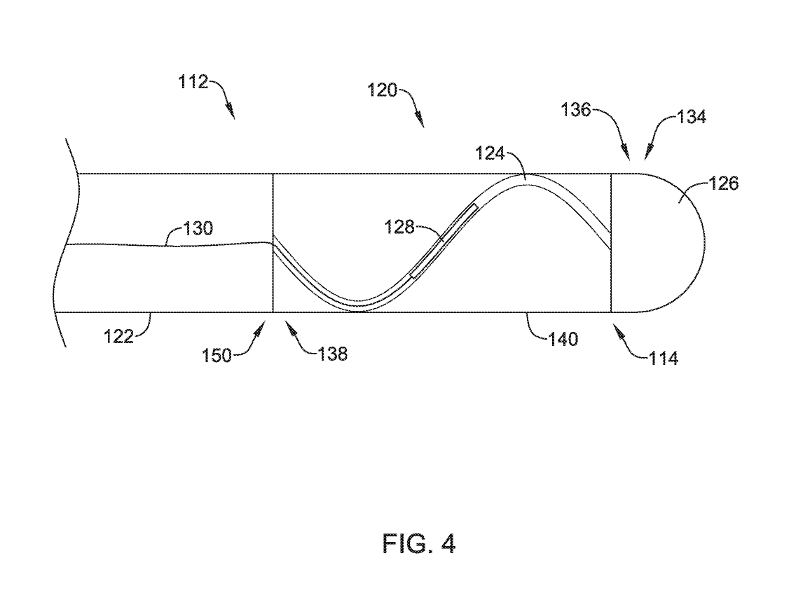

FIG. 4 shows another example of a distal region of a contact sensing catheter 112. In general, the construction and operation of contact sensing catheter 112 is similar to that of contact sensing catheter 12 shown in FIGS. 1-3. However, support member 124 of contact sensing catheter 112 is shown to be a helical member. Helical support member 124 may resemble a spring. Similar to that of contact sensing catheter 12, helical support member 124 may have a proximal end 138 and a distal end 136. The proximal end 138 of helical support member 124 may be coupled to the distal end 150 of catheter body 122. Similarly, the distal end 136 of helical support member 124 may be coupled to a proximal end 134 of distal tip 126. In some instances (for example, as shown in FIG. 4), support member 124 may resemble a coiled spring extending from the catheter body 122 to the distal tip 126. However, while FIG. 4 depicts support member 124 as a coiled spring, it is contemplated that support member 124 could be a variety of shapes, sizes, geometries or materials. For example, helical support member 124 could be a single tubular member having a helical cut along its length and spanning the gap from the catheter body to the distal tip. In other instances, the support member 124 may be formed of a tubular member having a plurality of discontinuous cuts or slots extending through the sidewall of the tubular member to provide the tubular member with a degree of lateral flexibility.

Similar to FIGS. 1-3, FIG. 4 shows polymer member 128 disposed along helical support member 124. In some instances, such as that shown in FIG. 3, polymer member 128 may be affixed along a surface of support member 124. However, it is contemplated that polymer member 128 may be coupled to support member 124 using a variety of methodologies. As discussed herein, polymer member 128 may be described as being “affixed,” “on” and/or otherwise embedded and/or encased on any structure contemplated herein. This is not intended to be limiting. Rather, polymer member 128 can be positioned and/or otherwise located at any suitable position/location along support structure 120, helical support member 124, along the catheter 112 and/or any other catheter structure. Positioning/locating polymer member 128 may include embedding, partially embedding, encasing, partially encasing, isolating, attaching, affixing, fastening, bonding to the outer surface, embedding within the wall, or the like. Additionally, it is contemplated that more than one polymer member 128 may be affixed to helical support member 124. For example, multiple polymer members 128 may be spaced along helical support member 124. In some instances, multiple polymer members 128 may provide independent inputs to processor 32. In other instances, multiple polymer members 128 may operate collaboratively to provide a single input to processor 32.

Polymer members 128 may be an EAP. In some instances, an EAP may output an electrical signal in response to a change in the polymer's shape, resistance, or the like. For example, in some instances the contact catheter's distal end region 114 may bend or deflect as a clinician manipulates the catheter inside a heart chamber. The deflection may result in a deflection of at least a portion of the helical windings of the helical support member 124, including polymer member 128. In the instance where the polymer member 128 is an EAP, polymer member 128 may output an electrical signal to processor 32. Polymer member 128 may include a variety of different materials. For example, polymer member 128 may include polyvinylidene fluoride.

In some instances, an EAP may output an electrical signal and/or surface charge in response to a change in the polymer's shape, resistance, mechanical properties, or the like. For example, in some instances the contact catheter's distal end region may bend or deflect as a clinician manipulates the catheter inside a heart chamber. The deflection may result in a deflection of support member 124. In instances where an EAP is affixed directly to support member 124, deflection of support member 124 may cause a corresponding deflection in the EAP. The deflection of the EAP may cause the EAP to output an electrical signal to processor 32. Processor 32 may receive the electrical signal transmitted from the EAP.

Similar to contact sensing catheter 12 of FIGS. 1-3, contact sensing catheter 112 may include a covering 140 extending over or along support structure 120. Covering 140 may be a coating, a solid tubular member or the like. In some instances, covering 140 may be attached to support structure 120. In other instances, covering 140 may extend from the distal end 150 of catheter body 122.

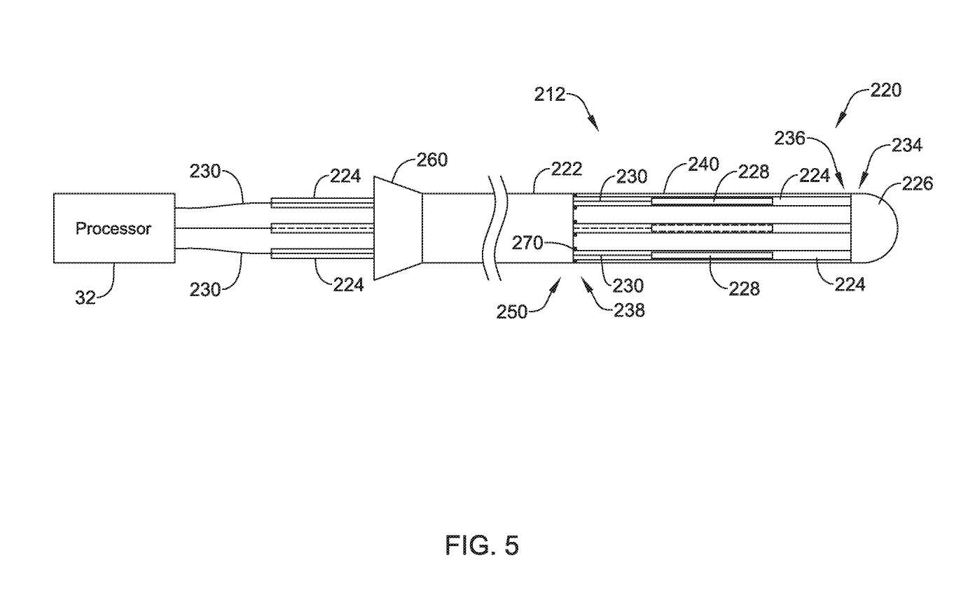

FIG. 5 shows another exemplary contact sensing catheter 212. Catheter 212 may include catheter body 222 and support structure 220. Support structure 220 may extend proximally from distal tip 226. Additionally, the distal end 236 of support members 224 may be coupled to the proximal end 234 of distal tip 226. Further, similar to other contact sensing catheter systems disclosed herein, support structure 220 may include one or more support members 224. Support members 224 may include polymer member 228. Polymer member 228 may include an EAP similar to other catheter sensing system embodiments disclosed herein. Support member 224 and/or polymer member 228 may be electrically coupled through conductive member 230 to processor 32. Further, distal tip 226 may include an electrode. The distal tip electrode may be an ablation electrode, a mapping electrode or some combination of an ablation and mapping electrode.

Support members 224 may extend along the outer surface, the inner surface or through the wall of catheter body 222. It is contemplated that support members 224 may extend proximally along a portion of, along substantially the entire length, or along the entire length of catheter body 222. It is contemplated that conductive member 230 may extend alongside support member 224 for the entire length of catheter body 222. While FIG. 5 shows catheter 212 having three support members 224, it is contemplated that the number of support members may be more or less than three. For example, support members 224 may include 1, 2, 3, 4, 5, 6, 7, 8, 9, 10, 20 or more members.

As stated above, support members 224 may extend proximally from distal tip 226. Further, in some instances support members 224 may be affixed to catheter body 222 at the distal end region 250 of catheter body 222. For example, support members 224 may be affixed to catheter body 222 at coupling region 270. While FIG. 5 shows support members 224 fixed to catheter body 222 at coupling region 270, it is contemplated that support members 224 may be fixed to catheter body 222 at other suitable locations along catheter body 222.

In some instances, support structure 220 (including support members 224) may deflect away from its equilibrium position when a force is applied to distal tip 226. Further, fixation of support members 224 to portions of the catheter proximal and distal of polymer member 228 permits axial elongation and/or axial compression of the support members 224. Therefore, a polymer member 228 (e.g. EAP) affixed to support members 224 may deflect in response to the same force experienced by the support structure 224. The degree of axial elongation and/or axial compression of polymer members 228 (e.g. EAP) may directly correlate to an electrical signal generated by the polymer member 228 (e.g. EAP). Further, this electrical signal may be utilized to calculate the magnitude and/or direction of the applied force.

It can be appreciated that whenever a force is applied to distal tip 226, the majority of the deflection seen is along a distal portion of the support members 224. Further, coupling support members 224 (to which force sensing EAP may be affixed) to catheter body 222 (e.g. at coupling region 270) reduces the lever arm of the deflectable portion of support members 224. Consequently, a force applied to distal tip 226 (causing deflection in EAP), may be more easily sensed. In other words, smaller deflections that might not otherwise be detected can now be sensed.

In some instances, support members 224 may be constructed separately from distal tip 226. In instances where support members 224 are constructed separately from distal tip 226, support members 224 may be coupled to distal tip 226 using a variety of manufacturing techniques. For example, distal tip 226 may be bonded to support members 224 using an adhesive. Other attachment methodologies are contemplated.

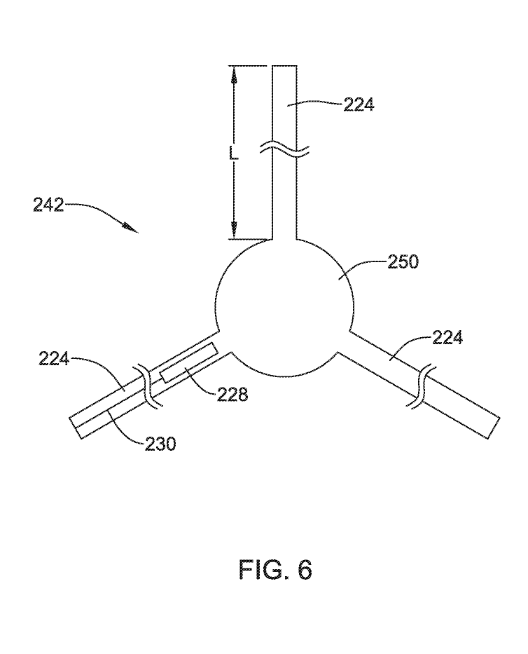

In some instances, support structure 220 (including support members 224 and/or distal tip 226) may be constructed and/or formed from a single monolith of material. For example, a method of manufacturing (e.g. cutting the material) support structure 220 (including support members 224 and/or distal tip 226) illustrated in FIG. 6 may bear some resemblance to an analogous processes utilized in the manufacturing of semiconductors.

The substrate utilized in the manufacturing process discussed above may include a flexible base material. In some instances, the material may include a polyimide sheeting, or other polymeric sheeting. Utilizing a flexible base material may provide desirable characteristics such as the ability to use the base material as the structural element itself. It is contemplated that the structural support 242 depicted in FIG. 6 could be directly shaped, manufactured, processed or configured to be integrated directly into a catheter sensing system. For example, FIG. 6 shows support members 224 lying in the same plane as hub member 240. FIG. 6 may resemble the configuration support members 224 have to hub member 240 immediately after support structure 242 is cut from a single monolith of material. However, further manufacturing may configure support members 224 such that they resemble the configuration of support members 224 and hub 240 displayed in FIG. 5. In other words, further manufacturing may bend and/or angle support members 224 to a position orthogonal to hub 240. It is contemplated that in some instances distal tip 226 may be manufactured separately from support structure 242. However, in other instances hub 240 may be substantially similar to distal tip 226.

In some examples herein, support member 224, alone or in combination with conductive member 230, may be referred to as a “printed circuit board.” Further, the manufacturing process described above may include “printing” or affixing conductive member 230 and/or polymer member along, atop, within, embedded with, etc. support member 224. For example, the example method of manufacturing may include forming a base layer of material upon which further layers may be “printed” or “layered.” The manufacturing method may further include layering one or more additional layers on top and/or within the base layer. Additional layers of material may include EAP's, electrically conductive materials, traces, circuit components, or the like. In some instances, a portion of a layer may be removed to expose an underlying layer. Further, the manufacturing process disclosed herein may contemplate incorporating sensors configured to sense a variety of inputs. For example, it is contemplated that temperature sensors, pressure sensors, ultrasound sensors, electrical impedance sensors and/or an ECG sensing element may be incorporated alone or in addition to any of the sensing members disclosed herein. Further, utilization of a flexible base material may enhance connectivity and provide for mounting of signal processing components (e.g. amplifier).

It is contemplated that any of the embodiments described herein may include a support structure including a rubber, plastic and/or polymer member. Support structures formed from these materials may provide desirable characteristics such as the ability to deform and/or deflection in response to a contact force. Similarly to embodiments described above, a rubber, plastic and/or polymer member may incorporate a polymer member such as EAP and/or derivatives thereof to elicit an electrical response upon a deformation and/or deflection due to contact force.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A system for sensing catheter contact, comprising:
an elongate tubular member having a proximal end and a distal end;
a tip member having a proximal end and a distal end;
a flexible support structure including a support member, wherein the support structure has a proximal portion and a distal portion, wherein the proximal portion is coupled to the distal end of the elongate tubular member, and wherein the distal portion is coupled to the proximal end of the tip member;
a polymer member disposed along the support member, the polymer member formed of an electroactive polymer, wherein displacement of the support member activates the polymer member such that an electrical response is output from the polymer member; and
a processor electrically coupled to the polymer member, wherein the processor is configured to evaluate the electrical response output from the polymer member to determine a contact force of the tip member with tissue.

2. The system of claim 1, wherein the flexible support structure includes a first support member, a second support member and a third support member, and wherein the polymer member is disposed along at least one of the first, second and third support members.

3. The system of claim 2, wherein the distal end of the tubular member has a substantially circular circumference, and wherein the first, second and third support members are spaced from one another around the circumference.

4. The system of claim 3, wherein at least one of the first, second and third support members include an arcuate bend.

5. The system of claim 4, wherein the first, second and third support members are configured to flex in response to the contact force.

6. The system of claim 4, wherein the elongate tubular member includes a longitudinal axis, and wherein the first, second and third support members include a proximal portion, an intermediate portion and a distal portion, and wherein the intermediate portions of the first, second and third support members extend inward toward the longitudinal axis relative to the proximal and distal ends of the first, second and third support members.

7. The system of claim 2, wherein the flexible support structure further comprises a printed circuit board, and wherein the printed circuit board includes the first, second and third support members.

8. The system of claim 7, wherein the printed circuit board includes a hub, and wherein a distal end of the first, second and third support members are coupled to the hub and wherein the proximal end of the first, second and third support members extends away from the hub.

9. The system of claim 8, wherein a polymer member is disposed along at least one of the first support member, the second support member and the third support member.

10. The system of claim 8, wherein the proximal ends of the first, second and third support members are coupled to the distal end of the elongate tubular member.

11. The system of claim 8, wherein at least one of the first, second and third support members extends along the elongate tubular member.

12. The system of claim 11, wherein at least one of the first, second and third support members extends to the proximal end of the elongate tubular member.

13. The system of claim 1, wherein the flexible support structure further comprises a printed circuit board, and wherein the printed circuit board includes a support member.

14. The system of claim 13, wherein the printed circuit board has a first layer, and wherein the first layer is an electroactive polymer.

15. The system of claim 1, wherein the flexible support structure includes a helical member, and wherein the polymer member is disposed along the helical member.

16. A system for sensing catheter contact, comprising:
a catheter having a proximal end region and a distal end region and a lumen extending therethrough, and wherein the catheter includes a flexible support structure disposed along the distal end region, and wherein the support structure includes a support member, and wherein the support member is configured to deflect in response to a contact force;
a polymer member disposed along the support member, the polymer member including an electroactive polymer, wherein deflection of the support member activates the polymer member such that an electrical response is output from the polymer member; and
a processor electrically coupled to the polymer member, wherein the processor is configured to evaluate the electrical response output from the polymer member to determine a contact force of the tip member with tissue.

17. The system of claim 16, wherein the flexible support structure is disposed within the lumen of the catheter.

18. The system of claim 17, wherein the flexible support structure includes a first support member, a second support member and a third support member and wherein a first polymer member, a second polymer member and a third polymer member are disposed along the first, second and third support members.

19. The system of claim 18, wherein at least one of the first polymer member, the second polymer member and the third polymer member is configured to determine the magnitude and/or the direction of the contact force.

20. A system for sensing catheter contact, comprising:
an elongate tubular member having a proximal end and a distal end;
a tip member having a proximal end and a distal end;
a first support member, a second support member and a third support member, wherein the support members have a proximal portion and a distal portion, wherein the proximal portions are coupled to the distal end of the elongate tubular member, and wherein the distal portions are coupled to the proximal end of the tip member;
a first polymer member disposed along the first support member, a second polymer member disposed along the second support member and a third polymer member disposed along the third support member, wherein the polymer members are formed of an electroactive polymer, wherein displacement of the support members activates the polymer members such that an electrical response is output from the polymer members; and
a processor electrically coupled to the polymer members, wherein the processor is configured to:
sense the electrical response output from the polymer members;
evaluate the electrical response to determine a magnitude and direction of the contact force; and
display the contact force magnitude and/or direction on a display.
